(19) United States
(12) Patent Application Publication
(10) Pub. No.: US 2021/0298765 A1
(43) Pub. Date: Sep. 30, 2021

(54) Thermal Necrosis Reducing Sawblade

(71) Applicant: Howmedica Osteonics Corp., Mahwah, NJ (US)

(72) Inventor: Carlos E. Collazo, Old Greenwich, CT (US)

(21) Appl. No.: 17/209,571

(22) Filed: Mar. 23, 2021

Related U.S. Application Data
(60) Provisional application No. 62/994,403, filed on Mar. 25, 2020.

(51) Int. Cl. A61B 17/14

(52) U.S. Cl. CPC A61B 17/14; A61B 2017/00849

(57) ABSTRACT

A corrugated sawblade includes a body extending from a proximal end to a distal end and a cutting element coupled to the distal end of the body. The body is corrugated such that the body forms a wavy pattern defining ridges creating a series of peaks and valleys along the body. The corrugated body reduces the surface area of the sawblade contacting surrounding surfaces such as a cutting guide and the interior of the bone through which the blade is cutting. The decreased surface area in contact reduces friction and therefore reduces the amount of heat generated by the oscillating motion of the blade, reducing the likelihood of osteonecrosis. The cutting surface coupled to the body has a rectangular cross section for producing a planar cut.

### CROSS REFERENCE TO RELATED APPLICATIONS

The present application claims the benefit of the filing date of U.S. Provisional Patent Application No. 62/994,403 filed Mar. 25, 2020, the disclosure of which is hereby incorporated by reference.

### BACKGROUND OF THE INVENTION

Oscillating saw blades are well known in the art and used to produce planar cuts, such as the femoral, tibial and patellar cuts utilized when performing total knee arthroplasty. While the use of oscillating saw blades is reliable, predictable and efficient, they do have undesirable side effects. To maximize the intimacy between the bone and implant in a total knee arthroplasty, cutting guides are used to abut the sawblades to produce a flat, planar cut. Existing sawblades are generally planar and guided by complementary planar cutting guides, thereby maximizing the contact surface area between the sawblade and cutting guide. Similarly, the large contact surface between the sawblade and the bone also results in a high friction environment during use. The thickness of the cutting teeth is generally the same as the sawblade body causing the sawblade body to rub against the surrounding bone as it follows the teeth. Additional friction is generated by the particulate bone debris that lodges between the blade and adjacent bone during cutting. Due to the high speed oscillation of the power drive and the resulting friction caused between the sawblade-to-bone and sawblade-to-cutting guide interfaces, excessive heat is generated. Such excessive heat is known to cause necrosis of the surrounding bone cells (i.e., osteonecrosis).

Osteonecrosis can have a substantial effect on the bone-implant interface in a total knee arthroplasty potentially resulting in aseptic loosening of the implant. Bone temperature should be maintained below 47° C. (116.6° F.) during cutting to avoid necrosis of the bone, but the friction caused by various surfaces of the sawblade often causes temperatures to exceed this threshold. One common practice to reduce heat is the use of saline irrigation, but this does not address the fundamental source of heat generation (i.e., friction). Therefore, further improvements are desirable.

### BRIEF SUMMARY OF THE INVENTION

The present invention pertains to medical devices for severing/cutting/resecting bones and other anatomical structures (e.g., veins, arteries, soft tissue, bowels, etc.). In particular, the present invention pertains to medical devices that resect bones in preparation for placement of a prosthetic implant.

The present disclosure includes a sawblade with a corrugated body. A wave pattern design on the body of the blade reduces the surface area in contact with both the surrounding bone and cutting guide during cutting. Further, the space created between the peaks and valleys of the wave pattern creates channels that provide an egress pathway for the morselized bone debris to exit the cutting site as the blade is advanced through the bone. The wave pattern also adds to the stiffness of the blade, which is desired for accurate cuts and further allows the overall thickness of the saw blade to be reduced compared to existing planar sawblades. The sawblade includes cutting teeth attached to the corrugated body that are configured to produce flat, planar cuts.

In certain preferred embodiments, the sawblade may comprise a body extending in a longitudinal direction from a proximal end to a distal end between first and second planes extending parallel to the body, the body being corrugated such that the body defines a first set of peaks cresting in a first direction orthogonal to the longitudinal direction, the body further defining a second set of peaks cresting in a second direction opposite the first direction, and a cutting element coupled to the distal end of the body wherein the cutting element defines a cutting surface having a rectangular profile configured to produce a planar cut. Each peak in the first set of peaks may be tangent to the first plane. Each peak in the second set of peaks may be tangent to the second plane. The cutting element may include a plurality of cutting teeth extending distally from the body. The cutting teeth may define the rectangular profile of the cutting element. The proximal end of the body may include a flat portion configured to couple to a power saw. The proximal end of the body may define a bore configured to receive a means for detachably coupling to a power saw. The body may be corrugated such that the first and second sets of peaks extend along the body in a lateral direction orthogonal to the longitudinal direction. The body and the cutting element may be monolithic. The body and the cutting element may be detachable. The body may define a center axis from which the first and second sets of peaks extend, and the first and second sets of peaks may be approximately equidistant from the center axis. The body may have a width of about 0.4 inches to about 1.0 inch. The body may have a thickness measuring between 0.01 and 0.05 inches. The distance between the first plane and the second plane may measure between 0.04 inches and 0.09 inches. The first set of peaks may include peaks forming a rounded edge. The first set of peaks may include peaks forming an angular edge. The thickness of the body may change as the body extends in a lateral direction wherein the lateral direction is orthogonal to the longitudinal direction. The distance between the first set of peaks and the second set of peaks may change as the body extends in a lateral direction wherein the lateral direction is orthogonal to the longitudinal direction. The sawblade may include straight lateral edges parallel to the longitudinal direction. The sawblade may include lateral edges transverse to the longitudinal direction.

### BRIEF DESCRIPTION OF THE DRAWINGS

FIG. 1 is a perspective view of an oscillating bone saw with a corrugated sawblade attachment.

FIG. 2 is top view of the corrugated sawblade illustrated in FIG. 1.

FIG. 3A is a proximal-facing view of the cross-section of the corrugated sawblade in FIG. 2 identified by line A-A.

FIG. 3B is a distal-facing view of the cross-section of the corrugated sawblade of FIG. 2 identified by line B-B.

FIG. 4 is a front view showing the distal end of the corrugated sawblade of FIG. 2.

FIG. 5 is a schematic perspective view of a sawblade abutting the surface of a cutting guide.

FIG. 6 is a schematic side view of a sawblade disposed within an internal slot of a cutting guide.

FIG. 7 is a schematic side view of a sawblade cutting through a bone while abutting a cutting guide.

### DETAILED DESCRIPTION

The present disclosure will now be described more fully hereinafter with reference to the accompanying drawings, in which some, but not all aspects of the disclosure are shown. Indeed, the disclosure may be embodied in many different forms and should not be construed as being limited to the aspects set forth herein. Like numbers refer to like elements throughout.

As used herein, the term “proximal,” when used in connection with a device or components of a device, refers to the end of the device closer to the user of the device when the device is being used as intended. On the other hand, the term “distal,” when used in connection with a device or components of a device, refers to the end of the device farther away from the user when the device is being used as intended. As used herein, the terms “substantially,” “generally,” “approximately,” and “about” are intended to mean that slight deviations from absolute are included within the scope of the term so modified.

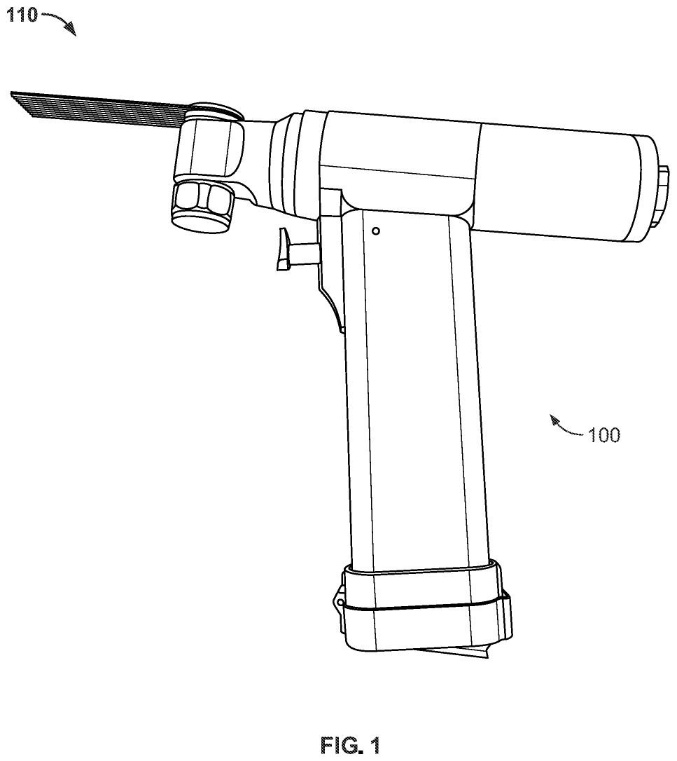

FIG. 1 is a perspective view of an oscillating bone saw 100. The illustrated bone saw 100 is a handheld motorized saw wherein a sawblade 110 is configured to oscillate in an orbital or side-to-side motion within a plane upon compression of a trigger. It is also contemplated that sawblade 110 can be coupled to a saw of any size, powered electrically or manually. The present disclosure relates to an improved sawblade 110 design which will be described below in further detail.

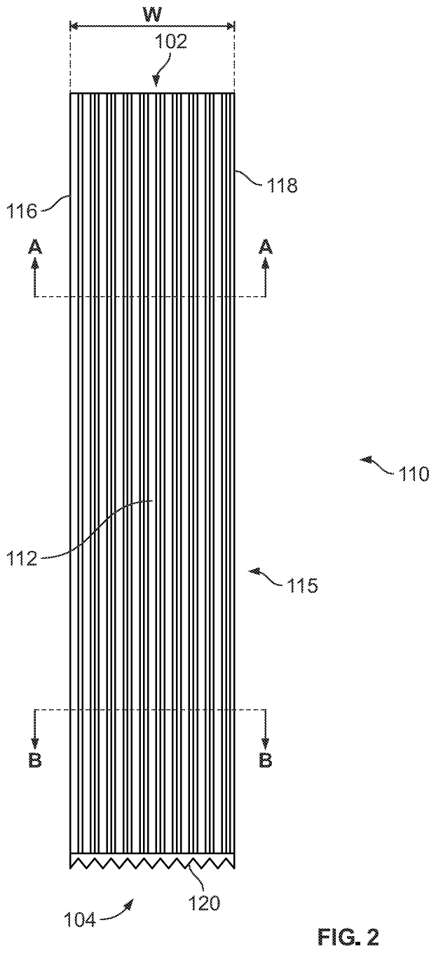

FIGS. 2-4 depict the sawblade 110 attached to the bone saw 100 in FIG. 1. The sawblade 110 extends from a proximal end 102 to a distal end 104 and between a first lateral edge 116 and a second lateral edge 118. In the illustrated embodiment, sawblade 110 has a corrugated body 115. Whereas a standard flat sawblade includes an entirely flat surface on its top and bottom surfaces, sawblade 110 comprises a wavy pattern having a top surface 112 and a bottom surface 114 that undulate as sawblade 110 extends from first lateral edge 116 to second lateral edge 118, as discussed further below. Proximal end 102 of sawblade 110 is configured to couple to a motorized device to generate a rapid oscillating motion in the lateral direction. Proximal end 102 of sawblade 110 may include a flat surface configured to fit within a clamp for coupling to bone saw 100. Proximal end 102 of sawblade 110 may include a bore or a fastening means for coupling to bone saw 100. Alternatively, proximal end 102 may couple to a handle to be held directly by a user for manual oscillation. At the distal end of corrugated body 115, sawblade 110 transitions into a flat row of teeth 120, which will be discussed below in further detail. In the illustrated embodiment, the first lateral edge 116 and the second lateral edge 118 are substantially straight. It is further contemplated that sawblade 110 may form any shape, wherein the first and second lateral edges 116, 118 include any angles or degrees of curvature.

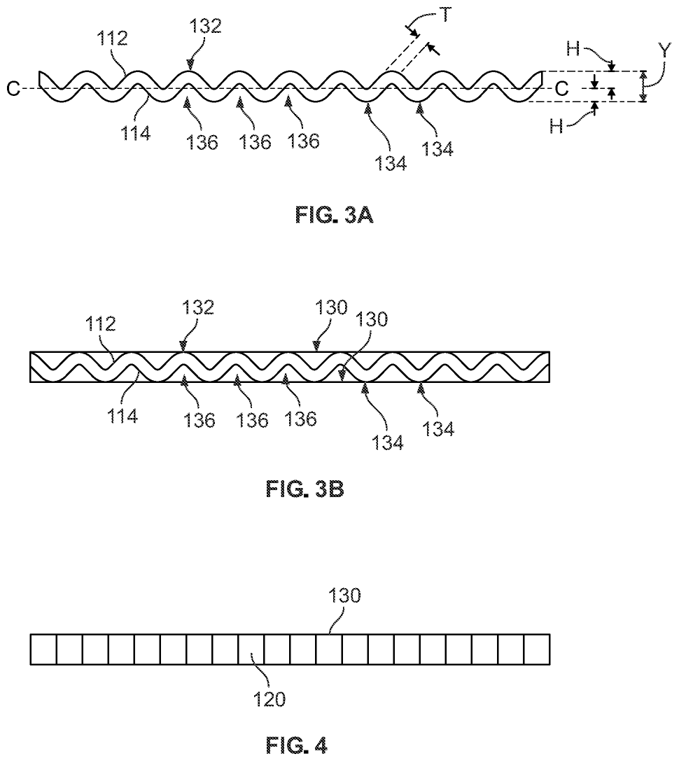

As illustrated in FIG. 3A, top and bottom surfaces 112, 114 undulate in sync with each other about a center axis C-C to form corrugated body 115, similar to a pair of sine waves. The undulation creates top surface peaks 132 and bottom surface peaks 134 along the width of sawblade 110, wherein the width, illustrated by “W” in FIG. 2, is defined as the distance between the first lateral edge 116 and the second lateral edge 118. The top surface peaks 132 and bottom surface peaks 134 each extend a height, wherein the height is shown by “H” in FIG. 3A and defined as the distance a peak 132, 134 extends from center axis C-C. In the illustrated embodiment, the height of each peak 132, 134 is consistent throughout the width of corrugated body 115 and the distance between all adjacent peaks 132, 134 is consistent. In this regard, the top surface peaks 132 are tangent to a first theoretical plane and the bottom surface peaks 134 are tangent to a second theoretical plane. The first and second theoretical planes are parallel to each other. It is contemplated that sawblade 110 may be constructed at any length and width combination with any number of peaks 132, 134, wherein the length is defined as the distance sawblade 110 extends from proximal end 102 to distal end 104. In FIGS. 3A-3B, the heights of peaks 132, 134 are equivalent, resulting in an equal number of top surface peaks 132 and bottom surface peaks 134. Heights and quantities of peaks 132, 134 may vary at the same proportion as illustrated or at different proportions. It is further contemplated that any variation in shape may be imposed on sawblade 110. For example, consecutive peaks 132, 134 may alternate heights wherein every other top surface peak 132 extends an equal height or every third top surface peak 132 extends an equal height, etc., or every peak 132, 134 may extend a different height from other peaks 132, 134, or the height of each peak 132, 134 may gradually increase or decrease across the width of sawblade 110. It is also contemplated that corrugated body 115 may undulate in a direction orthogonal to the direction illustrated in FIG. 2, such that top surface peaks 132 and bottom surface peaks 134 extend from first lateral edge 116 to second lateral edge 118 rather than proximal end 102 to the distal end of corrugated body 115.

The undulating nature of corrugated body 115 creates valleys between adjacent peaks 132, 134, which operate as bone debris egress channels 136. Egress channels 136 create space for bone debris to translate proximally along sawblade 110 to evacuate the interior of a bone as teeth 120 cut through bone and sawblade 110 advances distally. Evacuation of bone debris from cutting site further reduces heat generated from friction, as described below in further detail.

Distal end of corrugated body 115 includes a transition zone wherein sawblade 110 includes cutting element 130. FIG. 3B further shows cutting element 130 which comprises a plurality of cutting teeth 120. Teeth 120 are not corrugated, but configured in a straight alignment to create a flat, planar cut through a bone. Teeth 120 collectively form cutting element 130 having a rectangular profile as illustrated in FIGS. 3B and 4. The transition zone can be manufactured by any appropriate means known in the art, such as, e.g., standard milling processes or form stamping as is common in the sheet metal industry. Corrugated body 115 and teeth 120 may be manufactured monolithically or as separate attachable and detachable pieces.

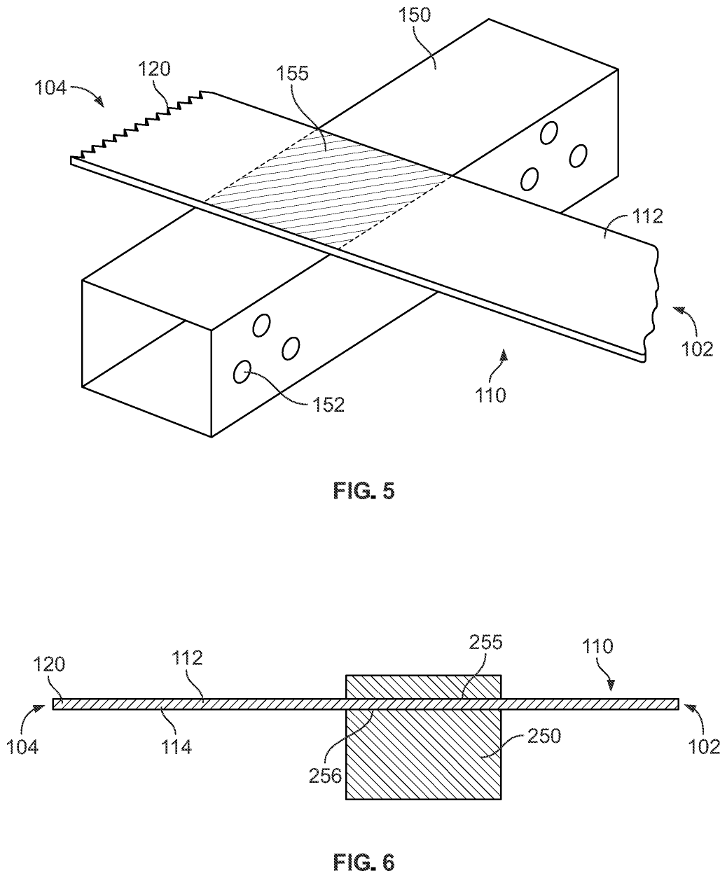

As discussed with regard to FIGS. 5-7, the illustrated design, and any alternate embodiments thereof, reduces the amount of heat generated through friction during the cutting process and increases the stiffness of sawblade 110 as compared to conventional saw blades. Frictional forces are reduced because sawblade 110 has a smaller surface area contacting surrounding surfaces relative to a sawblade with a flat body. As shown in FIG. 5, cutting guides 150 are often used to improve the stability of sawblade 110 while cutting to produce a flat, planar cut. As such, cutting guide 150 may be anchored into or adjacent a bone using a fastening means secured into anchor holes 152. With cutting guide 150 secured and sawblade 110 oscillating back and forth in a lateral direction (e.g., orthogonal to the proximal-distal direction of sawblade 110), friction is present between sawblade 110 and cutting guide 150 along a contact area 155. When a conventional flat sawblade is used, friction exists at every point of contact area 155. However, when corrugated sawblade 110 is used, friction no longer exists at every point of contact area 155. Friction is present only along the segments of sawblade 110 where bottom surface peaks 134 contact cutting guide 150. Thus, relative to a standard flat sawblade, friction in the present embodiment is proportionally reduced by the remainder of the bottom surface 114 which does not contact cutting guide 150. In other words, the surface area of a flat sawblade contacting cutting guide 150 within contact area 155 would equal the product of length and width of contact area 150. Meanwhile, sawblade 110 only contacts cutting guide 150 along each bottom surface peak 134, which may be reduced to line-contact rather than area-contact thereby substantially reducing the contact between sawblade 110 and cutting guide 150. Reduction in contact between sawblade 110 and cutting guide 150 significantly reduces frictional forces and thereby reduces the amount of heat generated by friction. In this regard, the heat produced by such friction is controlled to be less than what is typical to produce necrosis of bone tissue (i.e., below 47 degrees Celsius).

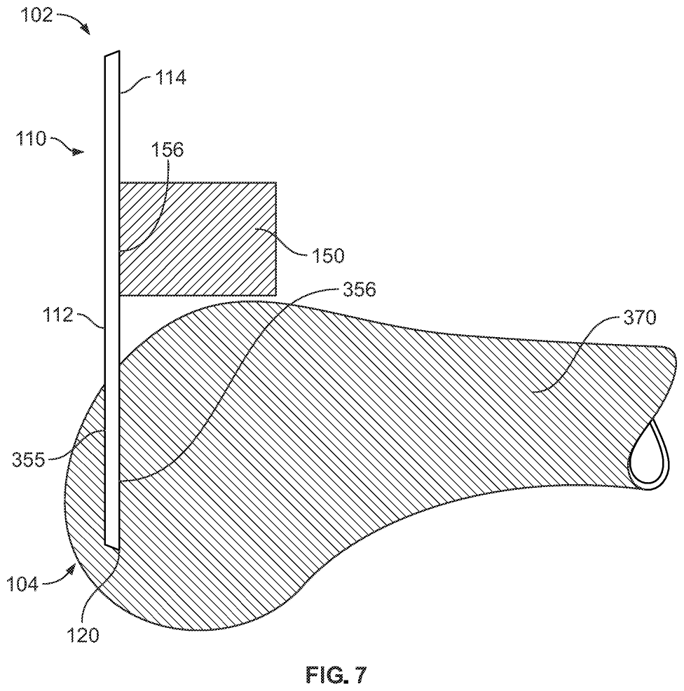

FIG. 6 illustrates a side view of sawblade 110 used with a slotted cutting guide 250 in which substantially the same principles apply as described above. Slotted cutting guide 250 may be used for additional stability when sawblade 110 is laterally oscillated to ensure a flat, planar cut in the bone. Slotted cutting guide 250 includes a slot in which sawblade 110 extends therethrough, wherein slotted cutting guide 250 abuts sawblade 110 along top surface 112 and bottom surface 114 of sawblade 110. Slotted cutting guide 250 includes space for sawblade 110 to move laterally (e.g., orthogonal to the proximal-distal direction) in both directions. Slotted cutting guide 250 and sawblade 110 form top contact surface 255 and bottom contact surface 256, which comprise the surface area of slotted cutting guide 250 in contact with sawblade 110. Thus, as sawblade 110 oscillates, friction exists along both top contact surface 255 and bottom contact surface 256. Substantially the same principles as described above in reference to cutting guide 150 in FIG. 5 apply to slotted cutting guide 250 in FIG. 6. Whereas a flat sawblade would contact slotted cutting guide 250 across the entirety of top contact surface 255 and bottom contact surface 256, corrugated sawblade 110 only contacts slotted cutting guide 250 where top surface peaks 132 contact slotted cutting guide 250 along top contact surface 255 and where bottom surface peaks 134 contact slotted cutting guide 250 along bottom contact surface 256. By significantly reducing the surface area in which sawblade 110 contacts slotted cutting guide 250, friction is significantly reduced and substantially less heat is produced during oscillation of sawblade 110.

FIG. 7 illustrates a side view of sawblade 110 and cutting guide 150 in use to cut a bone 370. Bone 370 can include any bone, such as a femur or tibia for, e.g., a total knee arthroplasty. Teeth 120 are coupled to distal end 104 of sawblade 110 to engage bone 370 while sawblade 110 is oscillated. Cutting guide 150 is anchored to or adjacent bone 370 to stabilize sawblade 110 and ensure a flat, planar cut. Bottom surface 114 abuts cutting guide 150 for guidance. Proximal end 102 of sawblade 110 attaches to a motorized device to be oscillated linearly in a lateral direction. As described above with respect to FIG. 5, oscillation of sawblade 110 along cutting guide 150 creates friction, but friction is reduced and less heat is generated due to the corrugated design of sawblade 110. Teeth 120 along distal end 104 cut into bone 370 and sawblade 110 continues to oscillate as sawblade 110 advances distally into bone 370. As sawblade 110 advances distally, top surface 112 and bottom surface 114 begin to contact surrounding bone 370, which creates top contact surface 355 and bottom contact surface 356. Substantially similar to the principles described with respect to slotted cutting guide 250 in FIG. 6, frictional forces at top contact surface 355 and bottom contact surface 356 generate heat. With a standard flat sawblade typically used in the art, the flat sawblade body would have width and thickness dimensions equal to the teeth, thus the entire surface area of the body rubs against bone 370, generating a substantial amount of heat. Further, the particulate bone debris fills any pockets and further clogs any remaining space between a flat sawblade and bone 370, increasing the normal force applied to the flat sawblade, thereby increasing frictional forces and generating more heat. Excessive heat leads to necrosis of the surrounding bone cells. The corrugated design of sawblade 110 reduces all of the aforementioned sources of friction and therefore generates less heat than a flat sawblade. Similar to the slotted cutting guide 250, only top surface peaks 132 of sawblade 110 contact bone 370 at top contact surface 355, and only bottom surface peaks 134 of sawblade 110 contact bone 370 at bottom contact surface 356. Further, bone chip egress channels 136 as described in FIGS. 3A-3B create pathways for bone debris to translate proximally along sawblade 110 and evacuate the interior of bone 370. Clearance of bone debris eliminates the source of additional friction and heat generated by a standard flat sawblade.

The distance between top surface peaks 132 and bottom surface peaks 134 is illustrated by “Y” in FIG. 3A. The heights of top and bottom surface peaks 132, 134 are preferably H=Y/2, meaning that the height of top surface peak 132 is equal to the height of bottom surface peak 134. In one example, Y may be 0.050 inches such that the top and bottom surface peaks 132, 134 have an H=0.025 inches. The corrugation allows the thickness of the material itself to be small because the corrugations provide rigidity and strength to sawblade 110. Such thickness, shown in FIG. 3A by “T” may be 0.020 inches, for example. The preferred width (W) of sawblade 110 is approximately 0.512 inches. However, it is contemplated that any size and any dimensions in accordance with the above-detailed principles are appropriate for sawblade 110.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A sawblade for cutting bone tissue comprising:
a body extending in a longitudinal direction from a proximal end to a distal end between first and second planes extending parallel to the body, the body being corrugated such that the body defines a first set of peaks cresting in a first direction orthogonal to the longitudinal direction, the body further defining a second set of peaks cresting in a second direction opposite the first direction; and
a cutting element coupled to the distal end of the body wherein the cutting element defines a cutting surface having a rectangular profile configured to produce a planar cut.

2. The sawblade of claim 1, wherein the first set of peaks are each tangent to the first plane.

3. The sawblade of claim 1, wherein the second set of peaks are each tangent to the second plane.

4. The sawblade of claim 1, wherein the cutting element includes a plurality of cutting teeth extending distally from the body.

5. The sawblade of claim 4, wherein the cutting teeth define the rectangular profile of the cutting element.

6. The sawblade of claim 1, wherein the proximal end of the body includes a flat portion configured to couple to a power saw.

7. The sawblade of claim 1, wherein the proximal end of the body defines a bore configured to receive a means for detachably coupling to a power saw.

8. The sawblade of claim 1, wherein the body is corrugated such that the first and second sets of peaks extend along the body in a lateral direction orthogonal to the longitudinal direction.

9. The sawblade of claim 1, wherein the body and the cutting element are monolithic.

10. The sawblade of claim 1, wherein the body and the cutting element are detachable.

11. The sawblade of claim 1, wherein the body defines a center axis from which the first and second sets of peaks extend, and the first and second sets of peaks are approximately equidistant from the center axis.

12. The sawblade of claim 1, wherein the body has a width of about 0.4 inches to about 1.0 inch.

13. The sawblade of claim 1, wherein the body includes a thickness measuring between 0.01 and 0.05 inches.

14. The sawblade of claim 1, wherein the distance between the first plane and the second plane measures between 0.04 inches and 0.09 inches.

15. The sawblade of claim 1, wherein the first set of peaks includes peaks forming a rounded edge.

16. The sawblade of claim 1, wherein the first set of peaks includes peaks forming an angular edge.

17. The sawblade of claim 1, wherein the thickness of the body changes as the body extends in a lateral direction wherein the lateral direction is orthogonal to the longitudinal direction.

18. The sawblade of claim 1, wherein the distance between the first set of peaks and the second set of peaks changes as the body extends in a lateral direction wherein the lateral direction is orthogonal to the longitudinal direction.

19. The sawblade of claim 1, wherein the sawblade includes straight lateral edges parallel to the longitudinal direction.

20. The sawblade of claim 1, wherein the sawblade includes curved lateral edges transverse to the longitudinal direction.
